(19) United States
(12) Patent Application Publication
(10) Pub. No.: US 2025/0235640 A1
(43) Pub. Date: Jul. 24, 2025

(54) EVALUATION UNIT FOR A MEDICAMENT DISPENSER, AND KIT COMPRISING AN EVALUATION UNIT AND A MEDICAMENT DISPENSER

(71) Applicant: Aptar Radolfzell GmbH, Radolfzell (DE)

(72) Inventor: Joachim KÖRNER, Uhldingen-Mühlhofen (DE)

(21) Appl. No.: 18/699,724

(22) PCT Filed: Oct. 5, 2022
(86) PCT No.: PCT/EP2022/077730

§ 371 Date: Apr. 9, 2024

(30) Foreign Application Priority Data
Oct. 28, 2021 (EP) 21205377.1

(51) Int. Cl. A61M 15/00; A61M 11/00; A61M 15/08

(52) U.S. Cl. CPC A61M 15/0065; A61M 11/007; A61M 15/08; A61M 2205/3592

(57) ABSTRACT

Evaluation unit for a medicament dispenser, designed to be attached in or to the medicament dispenser. The evaluation unit collects and processes data in the context of the medicament dispenser. The evaluation unit has an energy source, an evaluation electronics system, and a switch via which the energy source is galvanically connected to the evaluation electronics system and is galvanically isolated from the evaluation electronics system. The switch has a button for applying a mechanical force for establishing the galvanic connection between the energy source and the evaluation electronic system. This button is provided in the region of a contact surface, such that contacting the medicament dispenser applies force to the button and establishes the galvanic connection.

### FIELD OF APPLICATION AND PRIOR ART

The invention relates to an evaluation unit for a medicament-dispenser and also to the combination of such an evaluation unit with a medicament-dispenser, preferably in the form of a jointly marketed set.

Evaluation units of the type in question here serve for attachment to medicament-dispensers. They may serve various purposes. The primary field of application is the monitoring of the usage of the dispenser, in particular the monitoring of the release of medicaments. In the simplest case, the usages can be counted, in order, for instance, to indicate the remaining amount of medicament in the dispenser on a display. More elaborate evaluation units possess a usually wireless interface by means of which they are able to pass on data concerning the usage of the medicament-dispenser to other devices such as a smartphone or a server on the Internet.

Depending upon the type of monitoring, the evaluation units have to be permanently energized, at least in some cases, in order to be able to record a usage of the medicament-dispenser. This gives rise to a permanent consumption of power, which, in turn, rapidly drains a battery of the evaluation unit.

From various areas of technology it is known to separate a battery of a device in the delivery state from the electronical loads by means of an insulating strip. This strip is pulled out when the device is put into operation, so that the electronic loads are then connected to the battery. However, frequently this can only be implemented structurally with difficulty, depending on the position of the battery. In addition, the connection of the battery to the electronic loads is preserved after initial operation. For evaluation units that are used in succession on several medicament-dispensers, this is a disadvantage, since these dispensers may consume power to a relevant extent, above all also in a later, uncoupled state after initial operation.

### OBJECT AND SOLUTION

The object of the invention is to make available an evaluation unit and the combination of this evaluation unit with a medicament-dispenser, which both lessen the problems that have been explained.

In accordance with the invention, for this purpose an evaluation unit for a medicament-dispenser is primarily proposed that is designed for attachment within or on a medicament-dispenser. The evaluation unit comprises an abutment face provided for at least intermittent abutment against a wall of the medicament-dispenser.

In order that the evaluation unit does not unintentionally become detached from the medicament-dispenser, it can be fastened in suitable manner, in particular via a clamp connection, a latched connection, a threaded connection or some other positive or force-closed connection technique. At least in the region of the abutment face or parts thereof, the evaluation unit bears against the medicament-dispenser in the coupled state.

The evaluation unit comprises electronic components, in order to evaluate the handling of the medicament-dispenser. These components include an energy-source in the form of a battery, in particular a rechargeable battery, and also an electronic evaluation module. The evaluation electronic comprises at least one integrated circuit, in particular a microprocessor, and also at least one sensor. The sensor may be an integral part of the circuit or of the microprocessor or may take the form of a separate sensor unit that via inputs of the circuit is capable of being read out by the latter. For the purpose of storing sensor data, the evaluation unit preferably has a memory which may be an integral part of the microprocessor.

The sensor or sensors can record various types and data. In particular, one or more sensors have been jointly designed to be able to record the handling of the medicament-dispenser and, in particular, the release of medicaments.

The type of sensor or sensors and the number thereof for recording the release depend primarily on the type of the medicament-dispenser. It may be a question, for instance, of a tactile sensor that detects when an actuating handle of the medicament-dispenser is pressed down. However, the invention is particularly expedient in the case of evaluation units that have at least one sensor that gives rise permanently to sensor data and requires a comparatively large amount of power to do so. Such sensors include, for instance, motion-sensors that perceive translational or rotational accelerations or motions, acoustic sensors, or microphones, that enable the detection of the handling of the medicament-dispenser on the basis of noises generated in the process, as well as flowmeters that record a flow-rate of a liquid medicament or a flow-rate of a quantity of air flowing into the liquid reservoir for the purpose of equalizing pressure.

In addition to the integrated circuit or microprocessor and the at least one sensor, the evaluation unit preferably comprises an interface, in order to be able to pass on recorded sensor data, or data derived therefrom, to external systems. Such an external system may be, for instance, a mobile device of the user such as a smartphone or a server that is accessible via the Internet. The interface may be, in particular, a wireless network interface such as Bluetooth, WLAN or GSM, 3G, 4G or 5G.

The evaluation electronics is galvanically connected to the integrated energy-source in the operating state and is supplied with electrical energy via this source.

However, this galvanic connection may have been disconnected and may also be disconnected after the connection has been established. The evaluation unit comprises a switch for this purpose. This switch has a button for applying mechanical force for the purpose of establishing the galvanic connection between the energy-source and the evaluation electronics. If no force is applied to the button of the switch, the button returns to an unactuated state, as a result of which the galvanic connection, and therefore the power supply of the evaluation electronics, is separated from the energy-source. The switch has preferably been formed in the manner of a push-button which returns to an separating end position automatically, in particular by means of force applied by a spring, if it is not acted upon by a force from outside in the direction of its connecting end position.

The button is located in the region of the abutment face. This means that in the coupled state of the evaluation unit it points in the direction of the medicament-dispenser and has a force applied to it by the outside thereof, so that the connection between the energy-source and the evaluation electronics is established. Until the evaluation unit is fastened to the medicament-dispenser for the first time, however, the energy-source and the evaluation electronics are separated, and no loss of electrical energy from the energy-source takes place. If the medicament-dispenser and the evaluation unit are disconnected from one another again, the switch reassumes its separating position, and the consumption of power is terminated.

Although it is conceivable, in principle, that parts of the evaluation electronics remain permanently connected to the energy-source in order to provide a basic functionality even if the evaluation unit has not been coupled with a dispenser, it is preferred that the evaluation electronics are completely separated from at least one energy-source of the evaluation unit when the button does not have a force applied to it and is consequently in the state separated from the dispenser, so that the evaluation unit is no longer coupled with any load. It is possible that the evaluation unit comprises a further energy-source, for instance in the form of the battery of a battery-backed real-time clock. In such a case, the aforementioned complete separation relates to the main battery of the evaluation unit-that is to say, the battery having the largest capacity. The evaluation unit preferably comprises only a single energy-source, and when the button has not been actuated this source is separated from all the loads of the evaluation electronics. The designated purpose of such a configuration may consist, firstly, in the fact that the evaluation unit that is separated from the medicament-dispenser in the delivery state loses no energy at all, or loses energy only to a very slight extent, until the operating state is established for the first time. Except for the attaching of the evaluation unit to the medicament-dispenser, the user does not have to perform any additional activation steps.

The further purpose may consist in lowering the loss of energy in the energy-source to zero or almost zero in the meantime, even when the evaluation unit is separated from the medicament-dispenser again after initial operation, for instance in order to be coupled again with a new medicament-dispenser at a later time.

It may be desirable for electrical energy still to be available for a brief period after the separation of the evaluation unit from the medicament-dispenser. This can be achieved, in particular, by an electrical temporary-storage unit being provided that is capable of being separated from the energy-source together with the evaluation electronics by means of the switch. Such a temporary-storage unit may be constituted, in particular, by a capacitor, but an additional rechargeable battery of comparatively low capacity may also be capable of being used here.

The purpose of such a temporary-storage unit may consist, in particular, in triggering concluding functions of the evaluation electronics in response to the separation, for instance in storing the time of the separation in the memory, or in communicating the separation of the evaluation unit from the medicament-dispenser via a wireless interface to an external device, for instance to a cell phone linked by Bluetooth or directly to a server on the Internet via a mobile radio network. The capacity of the temporary-storage unit is preferably such that the evaluation electronics can remain in operation for at least a further five seconds before being switched off for lack of electrical energy.

The structural design of an outer housing of the evaluation unit depends crucially on the type of the medicament-dispenser. The outer housing preferably has a shape that permits a tool-free coupling and decoupling with/from the medicament-dispenser.

A particularly preferred structural design of such a housing provides that a ring-shaped or bowl-shaped segment is provided which is pushed onto a part of the medicament-dispenser, in particular at a bottom of a liquid reservoir. The ring-shaped or bowl-shaped segment may be designed to enter into a clamping connection with the medicament-dispenser. A threaded connection or some other connection acting in positive manner is also conceivable.

In such a configuration, the abutment face is provided on an inner end face of the bowl or, in particular, preferably on an inner side of the ring or bowl. The inner side can come into contact with an outer surface of the medicament-dispenser, such as, in particular, a cylindrical outer surface of a liquid reservoir of the medicament-dispenser, in which case the switch is pressed in or thrown so that the power supply of the evaluation electronics is established.

In particular, the evaluation unit as a whole may have a ring-shaped or bowl-shaped form. However, configurations are also conceivable in which at least one further functional element, for instance a sensor arm, extends away from the ring or bowl from the ring-shaped or bowl-shaped outer shape. A local thickening of a ring-shaped structure for the purpose of creating a receiving chamber for electronic components may also be expedient.

An evaluation unit of the described type may be a separately marketed product, in which case configurations are even possible in which the evaluation units are designed not for a specific medicament-dispenser but as generic evaluation units that can be attached to various types of dispenser.

However, it is particularly preferred if the evaluation unit is designed for a specific type of medicament-dispenser and is preferably part of a set that comprises at least one such medicament-dispenser and also the evaluation unit adapted thereto.

The evaluation unit is, however, preferably not absolutely essential for the operation of the dispenser. In particular, the evaluation unit is preferably provided for attachment to the medicament-dispenser such that no part of the evaluation unit comes into contact with the medicament in the course of discharge and consequently does not perform a liquid-conducting function.

The medicament-dispenser is usually a mobile medicament-dispenser which in the filled state usually weighs less than 500 grams, in particular less than 100 grams. It may be a question of a medicament-dispenser for medicaments in solid form, for instance for powders or tablets. In particular, however, the usage with medicament-dispensers is preferred for liquid medicaments that are emitted, in particular, in the form of a non-atomized jet, a spray jet or in the form of individual drops. The liquid reservoir of such a medicament-dispenser preferably accommodates a quantity of liquid of less than 100 ml, in particular preferably less than 50 ml.

The medicament-dispenser usually comprises a receiving chamber for a medicament prior to emission, constituted, in particular, by a bottle-like liquid reservoir. Furthermore, the medicament-dispenser comprises an extraction opening through which the medicament can be output, in particular by utilizing an extraction mechanism such as a pump or a squeeze-bottle configuration of the liquid reservoir.

The evaluation unit is designed in the manner described above and designed for detachable, preferably in tool-free manner, coupling to or within the medicament-dispenser. In the coupled state of the evaluation unit to or within the medicament-dispenser, the button of the switch has a force applied to it by the medicament-dispenser, so that the galvanic connection between the energy-source and the evaluation electronics of the evaluation unit is established.

If the evaluation unit and the medicament-dispenser form a set and are delivered to the customer jointly, it may be an advantage if in the course of delivery they are already in a pre-coupling state in which they are detachably connected to one another but offset from one another in comparison with the completely coupled state, so that the switch has not yet been actuated and the galvanic connection between the energy-source and the evaluation electronics of the evaluation unit has not yet been established.

Starting from this pre-coupling state, the user merely has to press the evaluation unit and the medicament-dispenser against one another in order to establish the completely coupled state and thereby to create the connection between the energy-source and the evaluation electronics. In particular, the direction in which the evaluation unit and the medicament-dispenser are pressed against one another may correspond to the actuation direction, so that the user automatically establishes the completely coupled state and the galvanic connection at the same time when the medicament-dispenser is actuated for the first time.

In the coupled state, the evaluation unit may be provided, in particular, in the region of an actuation face of the medicament-dispenser. The at least one sensor may then serve for detecting the actuation of the actuation face, for instance by taking the form of a tactile sensor that has a force applied to it when the actuation face is actuated, or by taking the form of an acoustic sensor that detects characteristic noises when the actuation face is actuated, which are subsequently evaluated and identified.

Alternatively, the evaluation unit may be provided in the region of the extraction opening of the medicament-dispenser, in order to record the discharge taking place through this opening. For this purpose, the sensor may take the form of a flowmeter. Here too, an acoustic sensor that is able to detect noises that are characteristic of the discharge enters into consideration.

In other sensor configurations, for instance in the case of an evaluation unit that detects the current handling via a motion-sensor, the location of the attachment of the evaluation unit to the medicament-dispenser is of lesser importance.

The use of an evaluation unit according to the invention is considered expedient, in particular, with the following types of medicament-dispenser.

A first type of a medicament-dispenser that is preferably used in accordance with the invention is an inhaler, in particular in the manner of a metered-dose inhaler (MDI), which comprises an upwardly open receiving shank, for receiving a medicament container, and also a mouthpiece angled relative to the shank.

In this case, the evaluation unit can be attached, in particular, to the mouthpiece of the inhaler, in which case the switch is provided on the evaluation unit in such a manner that it is actuated by being pushed on in the region of the mouthpiece and guarantees the energizing of the evaluation electronics. The evaluation unit in this case may, in particular, have been configured in the form of a ring or tube.

Alternatively, the evaluation unit of annular shape may have been attached to a distal end of the aforementioned receiving shank, or the evaluation unit of ring-shaped or bowl-shaped form may have been attached to the distal end of the medicament container itself, pushed into the receiving shank. Its sensors are then preferably designed to detect the actuating force acting on the container.

The medicament-dispenser of a set according to the invention may also take the form of a drop-dispenser with a liquid reservoir and with a discharge head. In this case, the discharge head comprises a discharge opening with drop-forming geometry. The evaluation unit is attached to an outside of the liquid reservoir or of the discharge head, and in the activated state monitors the actuation of the dispenser, for instance by evaluation of the stream of air that continues to flow into the liquid reservoir after actuation of the dispenser. The liquid reservoir may take the form, in particular, of a squeeze bottle.

The medicament-dispenser may also take the form of a powder-inhaler. Such a powder-inhaler is designed with a housing and preferably with a swiveling swivel cover articulated thereto, which is capable of being swiveled for the purpose of clearing a mouthpiece and/or for the purpose of providing a dose of powder, the evaluation unit preferably having been pushed onto the housing on the outside.

A further type of the dispenser of a set according to the invention is a nasal dispenser with a nasal applicator and preferably with a swirl chamber for the swirling of liquid in the region of the discharge opening. The evaluation unit in this case has preferably been provided on an actuating trigger of the medicament-dispenser, in particular on an actuating trigger situated opposite the nasal applicator.

Irrespective of the concrete type of dispenser, a medicament-dispenser that is used in accordance with the invention preferably comprises an actuating trigger which is capable of being depressed in relation to a storage body in which the receiving chamber is provided. This depressing of the actuating trigger brings about an emission of the medicament, in particular the emission of a liquid medicament. But the emission of a metered quantity of a powder or of a tablet is also conceivable.

In such a medicament-dispenser there may be provision, in particular, that the evaluation unit has an annular segment which is circumferentially attached to the storage body and which has an actuating section projecting beyond the actuating trigger in the coupled state and which is capable of being depressed together with the actuating trigger in relation to the storage body. Upon actuation, a relative motion of the actuating section and of the annular segment accordingly occurs, which is capable of being detected via a tactile sensor.

An alternative configuration provides that such a medicament-dispenser with actuating trigger comprises a sleeve surrounding the storage body, which is grasped by the user as intended. The actuating trigger is capable of being depressed in relation to the sleeve. With such a configuration, the evaluation unit is arranged within the sleeve below the storage body, so that the storage body presses on the evaluation unit when the actuation takes place. The evaluation unit may have a puck-like shape.

In one configuration, the medicament-dispenser takes the form of a powder-inhaler, preferably with a swiveling swivel cover which covers a mouthpiece in a closed state. A powder-inhaler according to the invention preferably comprises individual compartments attached to a flexible strip, which are filled with powder, a further one of these individual compartments being opened with each usage, in order that the powder can subsequently be inhaled through a mouthpiece.

Such powder-inhalers are usually designed with a flat housing. The evaluation unit is preferably designed to grasp this housing on both sides, in particular in the region of the swivel cover. The switch, by means of which the battery and the evaluation electronics are capable of being connected and disconnected, is preferably provided on a housing section of the evaluation unit, so that the evaluation unit is supplied with electrical energy as a result of the evaluation unit being pushed onto the powder-inhaler.

A further type of a medicament-dispenser that is preferably used in accordance with the invention provides that the medicament-dispenser comprises a receiving chamber for the medicament prior to discharge, which is capable of being relocated as a whole in relation to the extraction opening and which is capable of being relocated by application of force to an actuation face contrary to a piston. In particular, this relates to medicament-dispensers that are usually known on the market under the appellation “unit dose” or “bidose” and that provide a single discharge or two discharges before the receiving chamber has been drained.

In such a medicament-dispenser, the evaluation unit may have been attached to the actuating trigger in the region of the actuation face and, in particular, may have the bowl shape already described. The evaluation unit is compressed when force is applied to the actuating trigger and therefore to the receiving chamber, this being detected via the at least one sensor. In the supplied state, preferably in the pre-coupling state described above, the evaluation unit is attached to the actuating trigger, so that the switch has not been actuated and the galvanic connection between the energy-source and the evaluation electronics has not yet been established.

Even though use in the case of medicament-dispensers is mentioned hitherto as a field of application, a configuration according to the invention of an evaluation unit is also possible in the case of use with other dispensers, in particular in the case of liquid-dispensers from the field of cosmetics and personal hygiene. Such evaluation units are also assigned to the complex of inventions described herein.

### BRIEF DESCRIPTION OF THE DRAWINGS

Further advantages and aspects of the invention arise out of the claims and the following description of preferred embodiments of the invention, which are elucidated in the following with reference to the figures.

FIGS. 1A and 1B show a first embodiment comprising an evaluation unit and a liquid-dispenser which, by way of example, takes the form of a drop-dispenser with squeeze bottle.

FIGS. 2A to 2C show a second embodiment in which the medicament-dispenser takes the form, by way of example, of a liquid reservoir, in particular a cream-dispenser.

FIG. 3 shows a third embodiment in which the medicament-dispenser likewise takes the form of a liquid-dispenser.

FIGS. 4A and 4B show a powder-inhaler with an evaluation unit.

FIGS. 5A and 5B show a liquid-dispenser taking the form of a unit-dose or bidose dispenser.

FIGS. 6A and 6B show an embodiment in which the medicament-dispenser takes the form of a metered-dose inhaler and the evaluation unit is provided in the region of a mouthpiece.

FIG. 7 likewise shows a medicament-dispenser taking the form of a metered-dose dispenser, the evaluation unit here having been attached to the container.

### DETAILED DESCRIPTION OF THE EMBODIMENTS

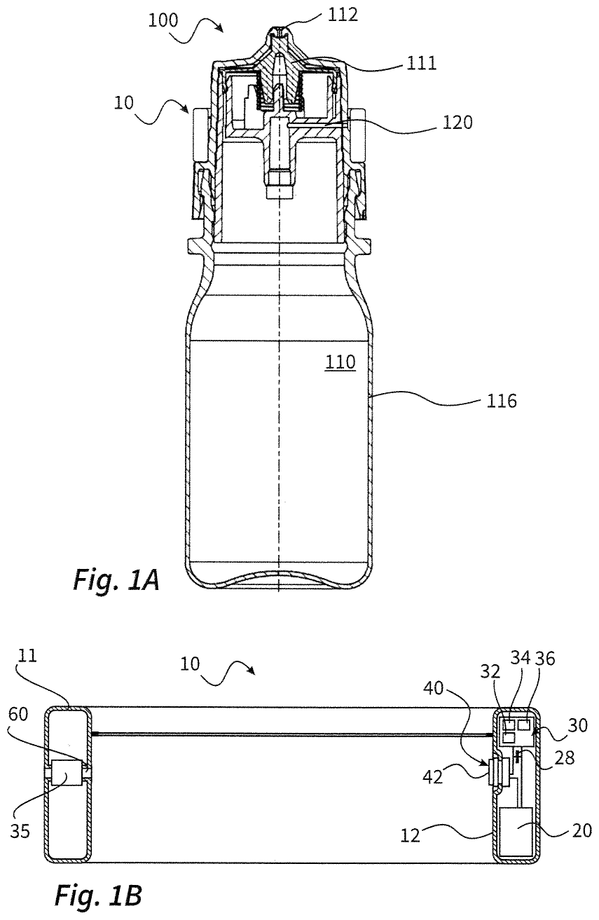

FIGS. 1A and 1B show a first embodiment of a medicament-dispenser 100 according to the invention with an evaluation unit 10. The medicament-dispenser 100 in the present case is a liquid-dispenser. The dispenser has a receiving chamber 110 serving as liquid reservoir, in which liquid medicament is stored prior to discharge. The liquid-dispenser 100 takes the form of a drop-dispenser and comprises a discharge opening 112 which is surrounded by drop-forming means, so that emerging liquid collects here before it is released from the dispenser in the form of a free-falling drop. An outlet valve 111 is positioned upstream of the discharge opening 112. For the purpose of actuating the liquid-dispenser 100, a reservoir body 116 made of elastically deformable synthetic material and surrounding the receiving chamber 110 is compressed. After the discharge, the reservoir body 116 returns to its initial position, with equalizing air being drawn in through a ventilation duct 120 for the purpose of avoiding a reduced pressure.

The evaluation unit 10 is provided on the discharge head of the liquid-dispenser 100. This evaluation unit 10 has an annular shape 11 and can be seen in sectional representation in FIG. 1B.

The functional principle of the evaluation unit 10 shown in FIG. 1B is based on the fact that this unit has an air-passage duct 60 which, in the coupled state of the evaluation unit 10, is positioned upstream of the ventilation duct 120 of the liquid-dispenser 100. Inflowing air for the purpose of equalizing pressure therefore has to flow through the air-passage duct 60. A flowmeter 35 is provided on the air passage duct 60, which permanently records how great the steam of air is through the air-passage duct 60 and, in particular, whether this stream of air lies above a defined threshold value. The flowmeter 35 is part of an evaluation electronics 30 which, in addition, also have further components such as a microprocessor 32, a motion-sensor 34 and also a wireless network interface 36.

So long as the evaluation unit 10 has not been coupled to the liquid-dispenser 100, a switch 40 provided on the inner abutment face 12 of the evaluation unit 10 in the form of a push-button with a button 42 has not been pressed in. By this means, the evaluation electronics 30, inclusive of the flowmeter 35, are galvanically separated from a battery 20.

Only when the evaluation unit 10 is pushed onto the dispenser 100 from above in the course of initial operation and is held there in clamping manner does the button 42 come into contact with an outer contour of the dispenser 100 and is pressed in by this means. By this means, a galvanic connection of the evaluation electronics 30 to the battery 20 is created, and consequently the power supply of the evaluation electronics 30 is established. From this point on, the flowmeter 35 has also been activated and is able to record the inflow of air into the liquid reservoir and, indirectly by this means, the previous extraction of liquid from the liquid reservoir. Corresponding data can be passed on via the network interface 36 to an external device or to a server on the Internet.

If the evaluation unit 10 is seperated from the dispenser 100, the battery 20 is also hereby separated from the evaluation electronics 30. In order nevertheless to be able to supply the evaluation electronics 30 with power for a few more seconds, a temporary current-storage unit 28 is provided which has a low capacity. It may be a question, in particular, of a capacitor. The capacity is sufficient to be able, for instance, to send a final data packet, in particular relating to the separation of the evaluation unit 10 from the dispenser 100, via the network interface 36.

If the evaluation unit 10 is attached again to a liquid-dispenser 100, the galvanic coupling is also created again by this means, and the evaluation electronics 30 are again supplied with power from the battery 20.

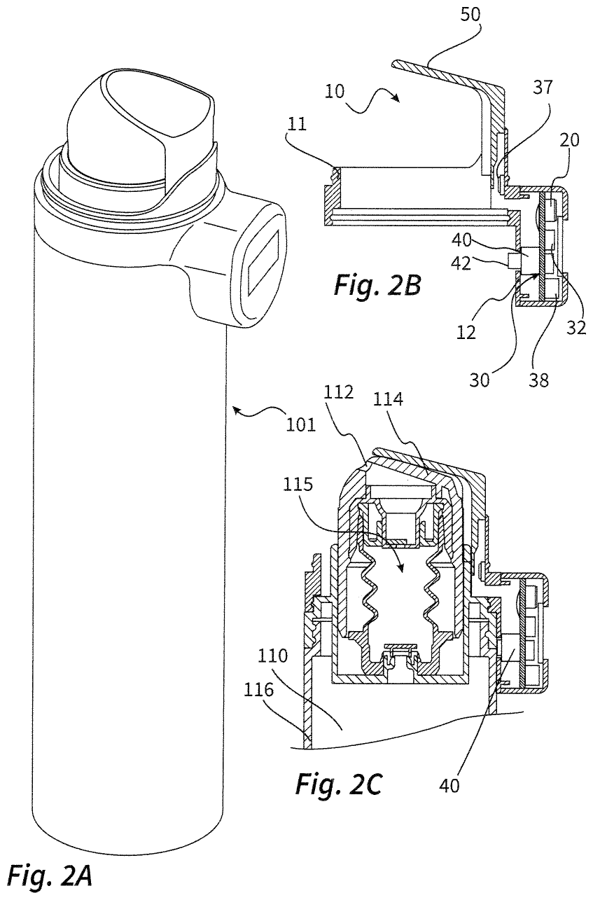

In the embodiment shown in FIGS. 2A to 2C, the medicament-dispenser 101 likewise takes the form of a liquid-dispenser. The dispenser has a receiving chamber 110 and also a pump device 115 which can be actuated via an actuating trigger 114 for the purpose of discharging liquid through the extraction opening 112. On the outside, the evaluation unit 10 represented in isolation in FIG. 2A is attached to the upper end of a storage body 116 of the dispenser 101. This unit has an annular segment 11 which is fastened surrounding the liquid-dispenser 101 and which has an actuating section 50 which extends upward from the annular segment 11 and over the actuating trigger 114. Also in this case, the evaluation electronics 30 have sensors 37, 38 which are connected to a microprocessor 32. Sensor 37 takes the form of a push-button which is triggered when the actuating section 50 is pressed down.

As in the case of the evaluation unit 10 shown in FIGS. 1A and 1B, here too a switch 40 is provided in the form of a push-button which is provided in the region of an abutment face 12 of the evaluation unit 10 and is pressed down by attachment of the evaluation unit 10 to the liquid-dispenser 101, as represented in FIG. 2C. In this depressed state, the switch connects the battery 20 to the evaluation electronics 30, in particular to the microprocessor 32 and to sensor 37. If the evaluation unit 10 is detached from the dispenser 101, the galvanic connection is interrupted, so that no relevant power consumption takes place.

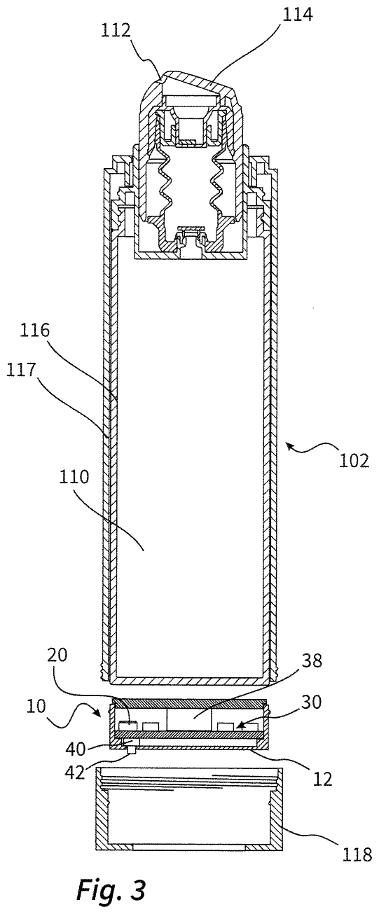

FIG. 3 shows a medicament-dispenser 102 which is largely identical to that represented in FIG. 2A. However, in addition to the components according to FIG. 2A, this dispenser has an elongated sleeve 117 which is capable of being sealed at the lower end by means of a stopper 118. If the dispenser 102 is actuated, this is done by the user enclosing the sleeve 117 with his/her hand and depressing the actuating trigger 114 with a finger. By this means, a force is generated between the storage body, which surrounds the receiving chamber 110, and the sleeve 117 as well as the stopper 118. For the purpose of detecting this, an evaluation unit 10 is provided which is inserted into the sleeve as intended, in the present case into the stopper 118 of the sleeve. This evaluation unit has on its underside a switch 40 with a button 42. If the evaluation unit, having approximately the shape of an ice-hockey puck, is pressed into the stopper 118, the switch 40 is activated and galvanically connects a battery 20 to an evaluation electronics 30 which have, in the manner already described, a sensor 38 and also a microprocessor 32 and a network interface 36. The sensor 38 is designed to detect the aforementioned force between the sleeve 117 and the storage body of the receiving chamber 110.

As soon as the evaluation unit 10 is removed from the stopper 118, the switch 40 is opened again in spring-propelled manner, so that the galvanic connection and the power supply of the evaluation electronics 30 are again disconnected from the battery 20.

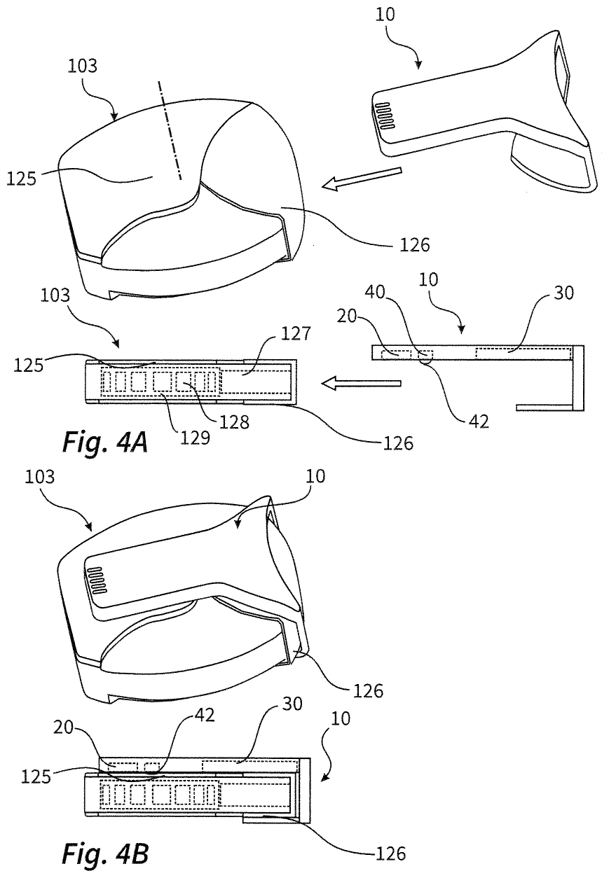

FIGS. 4A and 4B show a powder-inhaler 103 and also an evaluation unit 10 adapted thereto. In FIG. 4, the uncoupled state is represented. The powder-inhaler 103 pas a housing 125, within which a plurality of powder units are stored in individual compartments 128, the compartments being attached to a common flexible strip 129 which is present in the form of a reel and can be unwound therefrom.

A mouthpiece 127 is covered by a swivel cover 126. If this swivel cover is swiveled about the shown swivel axis, by this means the mouthpiece 127 is cleared and at the same time the flexible strip 129 is unwound a little and the compartment that is conveyed into an active position by this means is opened. The user can subsequently inhale the powder contained therein through the mouthpiece 127.

The evaluation unit 10 is intended to be pushed laterally onto the housing 125, in the course of which it does not impair the swiveling capability of the swivel cover 126. The evaluation unit is furnished with an evaluation electronics 30 which are fed with energy by a battery 20. A switch 40 taking the form of a push-button is provided on an inner side. Only in the pressed-in state does the switch 40 connect the battery 20 to the evaluation electronics.

If the evaluation unit is pushed onto the powder-inhaler 103 in the direction of the arrows, the button 42 of the push-button 40 comes into contact with the housing 125 and is pressed in by the latter. By this means, the evaluation electronics 30 are connected to the battery 20. This is represented in FIG. 4B.

From this point on, the evaluation electronics 30 are able to detect the opening of the swivel cover 126 and/or the inhalation itself, for instance via a microphone of the evaluation electronics or via a further push-button which in the closed state of the swivel cover 126 bears against the latter.

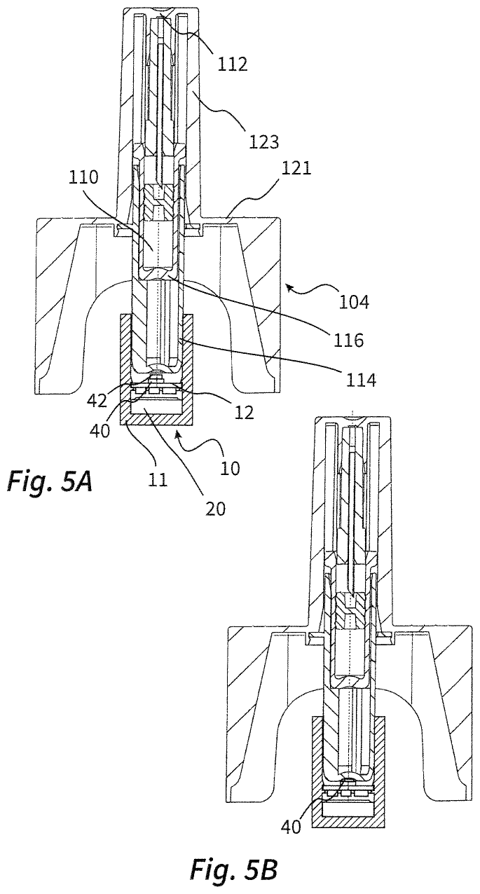

FIGS. 5A and 5B show a liquid-dispenser 104 which may be provided, in particular, for single or double use. The liquid-dispenser has a main body with applicator 123 and abutment faces 121 for index finger and middle finger, as well as an actuating trigger 114 by means of which a liquid reservoir with storage body 116 can be relocated upward so that a needle positioned there pierces a stopper in the receiving chamber 110. The liquid stored in the receiving chamber 110 can subsequently reach the extraction opening 112.

An evaluation unit 10 which has a bowl-shaped structure 11 is provided at the lower end of the actuating trigger 114. In the delivery state shown in FIG. 5A, the evaluation unit 10 is in a pre-assembly state in which a button 42 of a switch 40 is spaced from the lower end face of the actuating trigger 114. If force is now applied upward to the actuating trigger via the evaluation unit 10, the evaluation unit 10 is firstly relocated in relation to the actuating trigger 114, so that the switch 40 is closed and the evaluation electronics 30 are galvanically connected to a battery 20. From this point on, the evaluation unit 10 is able to monitor the handling of the dispenser 104—for instance, it can detect future actuations by means of a motion-sensor.

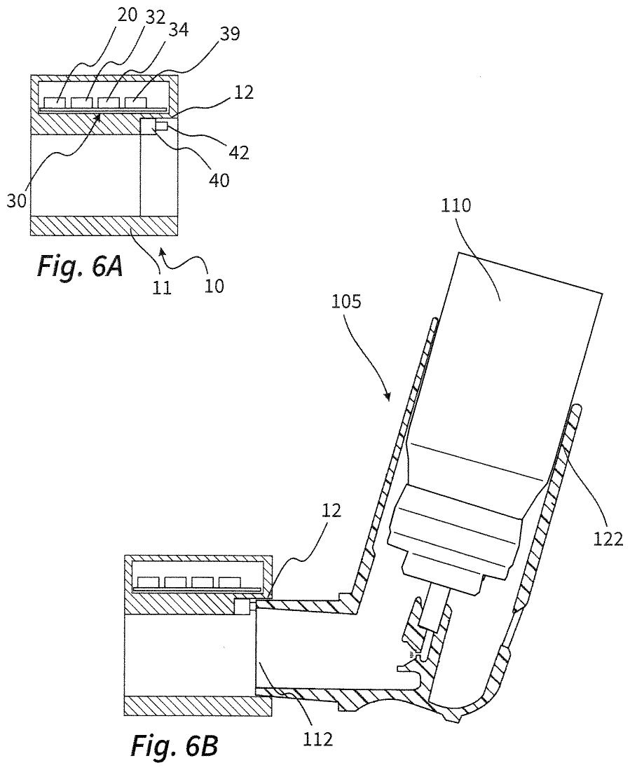

FIGS. 6A and 6B show a system in which the liquid-dispenser 105 takes the form of a metered-dose inhaler (MDI). This inhaler comprises an approximately L-shaped housing in which a container with receiving chamber 110 for a liquid medicament is provided in a receiving shank 122. The bottom of this container can be pressed down for the purpose of the discharge of liquid. By this means, a defined quantity of liquid is emitted at an outlet nozzle of the container and conveyed into a mouthpiece 112 of the liquid-dispenser 105.

The evaluation unit 10 represented in FIG. 6A is designed here to be fastened to the mouthpiece 112. When this fastening takes place, a switch 40 is actuated thereby, so that it connects a battery 20 to an evaluation electronics 30 of the type already described previously. The evaluation electronics 30 comprise a sensor 39 taking the form of a microphone. After energizing of the evaluation electronics 30, this sensor is able to detect a discharge process acoustically. If the evaluation unit 10 is disconnected from the dispenser 105, the switch also returns to its initial position, and the battery 20 is galvanically separated from the evaluation electronics 30.

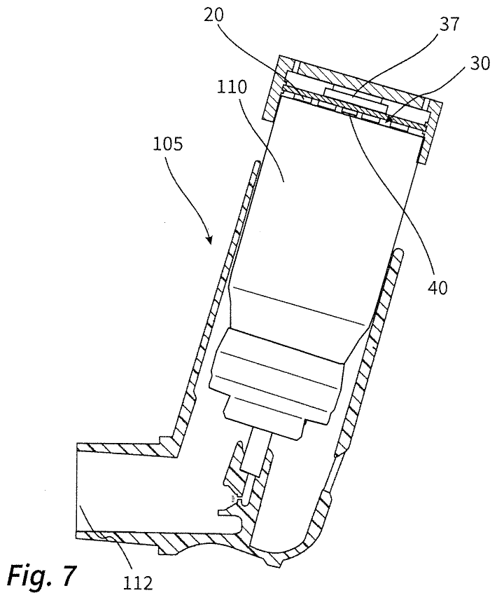

FIG. 7 shows an alternative configuration. Here, the evaluation unit 10 is attached to the bottom of the container. The evaluation unit 10 has a switch 40 which guarantees the energizing of the evaluation electronics 30 when the evaluation unit 10 is attached to the container. An actuation by pressing the container down can then be detected by virtue of the fact that a push-button 37 registers the depressing of an end face of the evaluation unit 10.

1. An evaluation unit for a medicament-dispenser, comprising:
an abutment face configured for abutment against a wall of the medicament-dispenser;
an electrical energy-source;
an evaluation electronics comprising at least one integrated circuit and at least one sensor; and
a switch galvanically connecting the energy-source to, and galvanically separating the energy-source from, the evaluation electronics
the switch having a button for application of mechanical force for establishing the galvanic connection between the energy-source and the evaluation electronics,
the button being located in a region of the abutment face such that the button has a force applied to it by abutment against the medicament-dispenser and in consequence of this the galvanic connection is established.

2. The evaluation unit as claimed in claim 1, wherein
the evaluation unit is configured for attachment to the medicament-dispenser such that no part of the evaluation unit comes into contact with the medicament during a discharge.

3. The evaluation unit as claimed in claim 1, wherein the energy-source comprises a sole energy-source, and
the evaluation electronics are completely separated—from the energy-source of the evaluation unit when the button does not have a force applied to it.

4. The evaluation unit as claimed in claim 1, wherein
the switch comprises a spring-loaded push-button, the spring-loaded push-button establishing the galvanic connection in a pressed-in state.

5. The evaluation unit as claimed in claim 1, wherein
the evaluation unit comprises an electrical temporary-storage unit, the electrical temporary-storage unit supplying the evaluation electronics with electrical energy even after galvanic separation from the energy-source.

6. The evaluation unit as claimed in claim 1,
wherein the evaluation unit comprises a ring-shaped or bowl-shaped segment, the abutment face and the switch being disposed on an inner side of the ring-shaped or bowl-shaped segment.

7. The evaluation unit as claimed in claim 1, wherein
the evaluation electronics comprises at least one wireless network interface.

8. A set comprising: a medicament-dispenser, the medicament-dispenser comprising a wall, a receiving chamber for storing a medicament prior to discharge and a discharge opening; and
an evaluation unit, the evaluation unit comprising:
an abutment face configured for abutment against the wall of the medicament-dispenser;
an electrical energy-source;
an evaluation electronics comprising at least one integrated circuit and at least one sensor; and
a switch galvanically connecting the energy-source to the evaluation electronics, the switch being separated from the evaluation electronics, the switch having a button for application of mechanical force for establishing a galvanic connection between the energy-source and the evaluation electronics, the button being located in a region of the abutment face, such that
in a coupled state of the evaluation unit on or within the medicament-dispenser, the button of the switch has a force applied to it by the medicament-dispenser, so that the galvanic connection between the energy-source and the evaluation electronics of the evaluation unit is established.

9. The set as claimed in claim 8, wherein
the medicament-dispenser and the evaluation unit are in a pre-coupling state in which they are already detachably connected to one another but are offset from each other in comparison with the coupled state, such that the galvanic connection between the energy-source and the evaluation electronics of the evaluation unit is not yet established, the switch being capable of having a force applied to it by the medicament-dispenser by application of force to the medicament-dispenser and to the evaluation unit, and the galvanic connection between the energy-source and the evaluation electronics is capable of being established.

10. The set as claimed in claim 8, wherein
the medicament-dispenser comprises a liquid-dispenser.

11. The set as claimed in claim 8, wherein:
the medicament-dispenser comprises an actuation face, and the evaluation unit is disposed in a region of the actuation face of the medicament-dispenser; or
the evaluation unit is disposed in a region of the discharge opening of the medicament-dispenser.

12. The set as claimed in claim 8, wherein
the medicament-dispenser is a metered-dose inhaler and an upwardly open receiving shank for receiving a medicament container, and a mouthpiece angled relative to the shank.

13. The set as claimed in claim 8, wherein
the medicament-dispenser is a drop-dispenser with a liquid reservoir and a discharge head, the discharge head comprising a discharge opening with drop-forming geometry, and the evaluation unit is attached to an outside of the liquid reservoir or of the discharge head.

14. The set as claimed in claim 8, wherein
the medicament-dispenser is a powder-inhaler with a housing and a swiveling swivel cover articulated thereto, the swiveling swivel cover being capable of being swiveled for clearing a mouthpiece and/or for providing a dose of powder.

15. The set as claimed in claim 8, wherein
the medicament-dispenser is a nasal dispenser with a nasal applicator and a swirl chamber configured for swirling liquid.

16. The set as claimed in claim 8, wherein
the medicament-dispenser comprises an actuating trigger capable of being pressed down with respect to a storage body, the receiving chamber being disposed in the storage body,

17. The set as claimed in claim 8, wherein
the medicament-dispenser is a Diskus inhaler.

18. The set as claimed in claim 8, wherein
the receiving chamber is relocatable as a whole in relation to the discharge opening and is relocatable by application of force to an actuation face contrary to a piston.

19. The evaluation unit as claimed in claim 7, wherein:
the at least one wireless network interface comprises a Bluetooth interface, a WLAN or GSM interface, a 3G interface, a 4G interface, or a 5G interface; and/or
the at least one sensor comprises a motion sensor; and/or
the at least one sensor comprises a sensor for recording a flow-rate of air or liquid; and/or
the at least one sensor comprises a microphone.

20. The set as claimed in claim 12, wherein the evaluation unit is attached to the mouthpiece, or the evaluation unit is attached to an upper end of the receiving shank or to the medicament container.

21. The set as claimed in claim 15, wherein the evaluation unit is attached to an actuating trigger of the medicament-dispenser disposed opposite the nasal applicator.

22. The set as claimed in claim 16, wherein:
the evaluation unit has an annular segment attached circumferentially to the storage body and an actuating section projecting beyond the actuating trigger in the coupled state, the actuating section being capable of being pressed down together with the actuating trigger relative to the storage body; or
the medicament dispenser comprises a sleeve surrounding the storage body, the actuating trigger being capable of being pressed down with respect to the sleeve, the evaluation unit being disposed within the sleeve below the storage body.

23. The set as claimed in claim 18, wherein the evaluation unit is attached to the actuating trigger in a region of the actuation face, the evaluation unit in the delivery state being attached to the actuating trigger such that the switch is not actuated and the galvanic connection between the energy-source and the evaluation electronics is not established.
